(19) United States
(12) Patent Application Publication
(10) Pub. No.: US 2002/0013519 A1
(43) Pub. Date: Jan. 31, 2002

(54) Secure test and test result delivery system

(76) Inventors: Scott Adams, Los Gatos, CA (US); Walter Wallach, Los Altos, CA (US)

Correspondence Address: Larry E. Vierra, Esq., VIERRA MAGEN MARCUS HARMON & DENIRO, Suite 540, 685 Market Street, San Francisco, CA, 94105-4206, US

(21) Appl. No.: 09/881,479

(22) Filed: Jun. 14, 2001

Related U.S. Application Data
(60) Provisional application No. 60/211,696, filed on Jun. 14, 2000.

(51) Int. Cl. A61B 5/00

(52) U.S. Cl. 600/300

(57) ABSTRACT

A system and method for a physician to cause a sleep test device to be delivered to a patient, for retrieving the collected data (and optionally the test device), and for the controlled and secure distribution of the results of the test collection to the physician. The system includes a user interface coupled to a network, including a test ordering system, a verification system and a database including patient, test and physician records; a shipping system, coupled to the ordering system, delivering the test apparatus to the patient; a retrieval system, coupled to the ordering system; and a result delivery system, delivering the results to the physician.

### BACKGROUND OF THE INVENTION

1. Field of the Invention

The invention relates to the collection and dissemination of confidential test data. In particular, the invention relates to the ordering of medical tests, delivery and retrieval of medical test devices, control of medical test information, and secure and efficient distribution of the information to authorized physicians.

2. Description of the Related Art

A number of medical tests require data collection from a patient over a lengthy period of time. Often, such tests must be performed in a controlled environment to ensure the accuracy of the test results. However, new tests are constantly being developed in areas traditionally requiring such laboratory testing which allow the patient to provide test data in a more comfortable environment, such as their home.

One particular field of medical testing where home testing can provide a significant advantage over traditional laboratory testing techniques is in the field of sleep apnea. Sleep apnea is among the most common and most dangerous types of sleep disorder. The condition is marked by repeated episodes of cessation of breathing during sleep that over time can lead to high blood pressure, cardiac disease, and disordered thinking. Obstructive sleep apnea is by far the most common type. Breathing is interrupted when air can't flow into or out of the nose or mouth. The reason for the blockage could be an over-relaxation of the throat muscles and tongue, which partially blocks the airway or, in obese people, an excess amount of tissue in the airway. In the less common form, central sleep apnea, breathing is stopped not because the airway is closed but because the diaphragm and chest muscles stop working. Traditionally, before being diagnosed with sleep apnea, a patient must submit to testing at a specialized sleep laboratory. The laboratory includes feedback equipment to measure a patient's physical reactions and brainwave activity during sleep testing which is performed at the site. The patient may be forced to attempt to sleep in a laboratory room with a bed which may be observed by the test technician. As such, this methodology is quite time consuming. Moreover, because of the limitations in the availability of facilities and trained technicians, the overall diagnosis can take several months: there is an initial delay in scheduling the laboratory time, followed by the all night or multiple night exam at the laboratory, further followed by several weeks delay in providing the results of the exam to the patient's physician.

When the test is completed, the results are analyzed and interpreted by a physician at the sleep lab. Typically this physician charges a fee for this over and above the fee for the test.

For the patient's physician, the current practice of referring a patient to a sleep lab often means that the patient comes under the care of the sleep lab physicians, and the physician referring the patient loses control of the patient. This presents a business problem for that physician and a discontinuity in patient care. If non-surgical treatments fail, the referring physician (often a surgeon specializing in pulmonology or otolaryngology) may or may not have an opportunity to treat the patient because the patient typically does not return to the referring physician who many times can best treat the patient.

Testing a patient for sleep apnea in a sleep lab is expensive and not necessarily representative of the patient's normal behavior. The expense presents a problem for patients who are paying for the test themselves, and increases the cost borne by the health insurance carriers.

As a result, devices have been developed which allow physicians to test for sleep apnea in the home. Devices such as those disclosed in U.S. Pat. Nos. 5,797,852 and 5,844,996 provide feedback to physicians following a period of in-home testing by a patient. Typically the patient has to go to the physician's office or sleep lab to have the test device placed on them, and the patient thereafter carries the device home for a one-night test. The test device is then hand-carried by the patient to the physician, who extracts data from the device and provides it to the physician who requested the test.

Delivery, retrieval and tracking of these test devices present logistical issues for a test facilitator. For example, a physician typically requests that a patient be tested by having the patient make an appointment to visit the sleep lab. There, the patient is physically connected to a test device, and sent home with wires attached to various parts of his or her body. The identity of the patient being tested is maintained through the patient carrying the device back to the sleep lab the next morning. The results of a test are typically mailed or sent by fax to the requesting physician's office. Thus, current practice does not facilitate ordering of sleep apnea tests for patients, nor does it facilitate delivery and retrieval of home testing devices or the identification of the patient whose test data is captured a particular device. Finally, current practice does not facilitate the secure delivery of confidential medical test results to everyone authorized by the patient to receive them.

Delivery issues exist with treatment as well as diagnosis. For example, the most common effective treatment for obstructive sleep apnea is nasal continuous positive airway pressure, or CPAP. The patient wears a soft plastic mask over his or her nose while sleeping. A device supplies pressurized room air through a flexible tube attached to the mask. The pressurized air acts as a stent to prevent the airway from collapsing. To prescribe CPAP treatment, the physician must first order a CPAP titration. The patient is typically sent back to the sleep lab for the titration step. If a CPAP is indicated, the patient is sent to a medical device vendor with a prescription for a device with the proper treatment setting. The patient then takes the device home with them. In some cases, the patient picks up a titration unit from a sleep lab in order to test it themselves at home. The patient returns this device, and its pressure readings are extracted and used for calibration and setting of the correct pressure for the CPAP machine.

Patients are typically seen initially by a primary care physician, who does not specialize in sleep disorders. This physician then refers the patient to a specialist, who manages the initial diagnosis. As explained above, the specialist often must refer the patient a second time to a sleep lab, which manages and evaluates the sleep test, and often prescribes the non-surgical treatment. The referral process usually involves paper forms. Presently, there is no process or capability available which simplifies this referral process for the primary care physician, and facilitates the specialist's sharing information with the primary care physician. This process gets more cumbersome when more than one specialist is involved (for example, a primary care physician with a patient with sleep apnea, and resulting cardiac problems).

There are other medical conditions where a patient must be monitored and data collected as they proceed with “normal” aspects of their lives. One example is cardiac monitors. These are ordered by a cardiac physician, and must be delivered to the patient, collected data must be retrieved and analyzed, and results must be distributed securely to the proper authorized individuals.

### SUMMARY OF THE INVENTION

The invention, roughly described, comprises a system and method for a physician to cause a sleep test device to be delivered to a patient, for retrieving the collected data (and optionally the test device), and for the controlled and secure distribution of the results of the test collection to the physician.

The invention further comprises a system for delivering an in-home test device to a patient at the request of a physician. The system includes a user interface coupled to a network, including a test ordering system, a verification system and a database including patient, test and physician records; a shipping system, coupled to the ordering system, delivering the test apparatus to the patient; a retrieval system, coupled to the ordering system; and a result delivery system, delivering the results to the physician.

Another embodiment of the invention includes a system for delivering a home test unit to a patient upon the request of an authorized physician, comprising: a test ordering system, including a telephone access system, a facsimile access system and a WWW access system; a data management system coupled to the ordering system; a test distribution and retrieval system coupled to the data management system; and a test data distribution system. The test data distribution system is coupled to the data management system; wherein the data management system further includes: an authorization system verifying the identity of the authorized physician; an order tracking system; and a device tracking system.

In yet another embodiment of the invention, a method is provided for delivering a test unit to a patient and distributing results of the test, comprising receiving an order from a physician for distribution of a test to a patient via a computer coupled to a network; verifying the authorization of the physician to place the order and the patient to receive the order; storing tracking information regarding a test unit shipped to the patient; shipping the test unit to the patient; retrieving test results from the unit; and distributing the test results.

In a further embodiment, the invention comprises a system for delivering a medical test to a patient and distributing the results of the test to a physician, comprising a World-Wide Web based portal including a secure test ordering interface, a database order processing system coupled to the portal, a logistics system interface, and a result distribution system.

In still another embodiment, the invention provides a World-Wide Web-based portal interface to a system for delivering a medical test to a patient and distributing the results of the test to a physician, comprising: a secure test ordering interface, and a secure test result retrieval interface.

### BRIEF DESCRIPTION OF THE DRAWINGS

The invention will be described with respect to the particular embodiments thereof. Other objects, features, and advantages of the invention will become apparent with reference to the specification and drawings in which:

FIG. 1 is a flow chart of a first embodiment of a process in accordance with the present invention.

FIG. 2 is a process-oriented block diagram of the operational components of the system of the present invention which implement the method of the present invention.

FIGS. 3A and 3B are flow diagrams demonstrating the process of the present invention as implemented in one embodiment of the system of the present invention.

FIG. 4 is a block level diagram of the data structure utilized in accordance with the system of the present invention and the tables found therein.

FIGS. 5-8 are use-case diagrams illustrating the portions of the system services which are available to different classes of authorized requestors in accordance with the system of the present invention.

### DETAILED DESCRIPTION

The present invention provides a unique system and method for secure home medical test ordering, test data collection, and secure distribution of collected test data to authorized patients and physicians.

The system includes software and hardware systems for order tracking, device tracking, data tracking, and interfaces to functional sub-systems. Such functional sub-systems may include courier services to provide distribution of tests from a central testing facility to patients in their homes, as well as information retrieval and reconditioning facilities staffed by individuals trained to perform specific functions described herein. The system can be adapted for use in contained environments, such as hospitals, to allow for test distribution to be distributed to patients' rooms, the results tracked, and the test results returned to the doctor in an efficient and more timely manner than has been performed in the prior art.

Process Overview

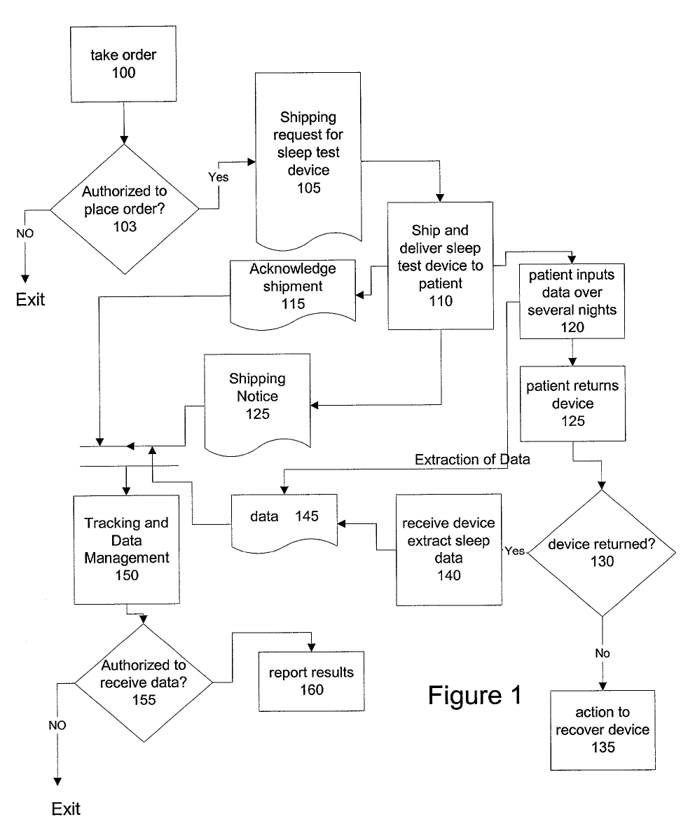

FIG. 1 shows a block level diagram of one implementation of the general method of the invention. Unless otherwise indicated, steps in the method are performed by a system administrator.

At step 100, an order for a sleep apnea test is received from a physician who is authorized to order a test to be prepared for a patient. The physician may input an order via a telephone, facsimile-transmitted order form, or using an Internet browser (using an encrypted connection via SSL, for example, for security).

After receiving an order at step 100, an authorization query step 103 determines whether the order for a test device is authorized. If the physician placing the order is not authorized at authorization step 103 the process terminates, or more information may be gathered in order for the physician to become authorized to order the data collection device. If the order is authorized, then a request to deliver a device (a shipping order) is created (step 105). Step 105 may involve additional steps (not specifically illustrated here, but described further below), involving preparing the device for shipment and communicating with a shipper to deliver the device to the patient.

At step 110, the device is delivered to the patient. The delivery step 110 can be performed by any form of courier service, including commercial courier services such as Federal Express® and/or United Parcel Service (UPS)®. The device may also be delivered to a “will-call” location for patient pickup, and returned by the patient to a similar “drop-off” location. The courier will pick from inventory and ship a collection device at step 110, providing an order acknowledgment 115 to the tracking and data management function 150. For simplicity, the same courier or shipping service would be used, with the return shipment prepaid.

An acknowledgment that the device is available for delivery and that the shipping order from step 105 has been received is provided at step 115 and returned to a tracking and data management 150. When the device actually ships, a separate shipping notice 125 is returned to tracking and data management 150. It should be noted that acknowledgement 115 and notice 125 are optional. After the patient receives the device, the patient performs the test at step 120. In one embodiment, data from the test may be extracted from the device and returned to the system administrator immediately following completion of the test.

The patient is responsible for returning the device through the appropriate mechanism, such as those described below, to the system administrator or a receiving agent in step 125. As further detailed, the return mechanism is arranged prior to delivery of the device to make the return of the device a simple matter for the patient. At decision step 130, a check is made to determine whether the device has, in fact, returned. If the device is not returned, at step 135 actions may be taken to recover the device. If the device is returned, at step 140, the device is received by the system administrator, or a designated receiving agent, and data from the test is extracted.

Test data 145 is returned to tracking and data management function 150. Data may be processed or analyzed at this time, or data may be processed or analyzed prior to reporting. Subject to ensuring that a recipient is, in fact, authorized to receive test results (decision box 155), the results may be distributed at step 160 by the system administrator. Actual results are always under the control of the system administrator. An alternate data extraction path is shown from patient inputting data in step 120 directly to the tracking and data management 150 (data 120), bypassing the physical return of the device (steps 125, 130, 140).

Delivery and Retrieval System

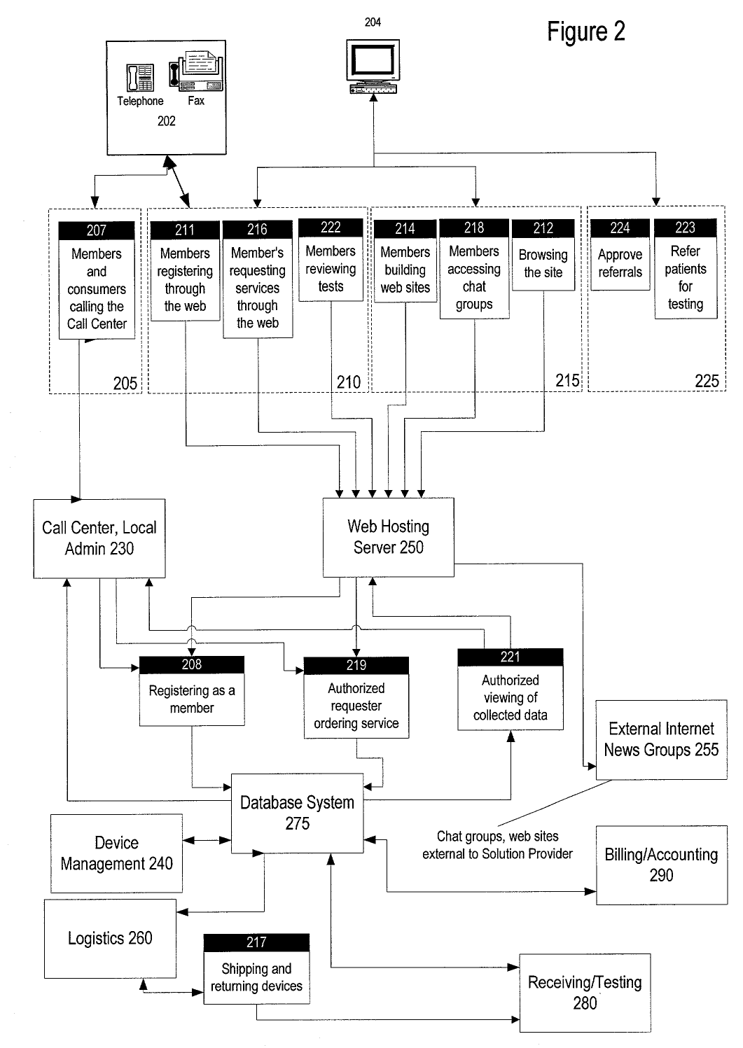

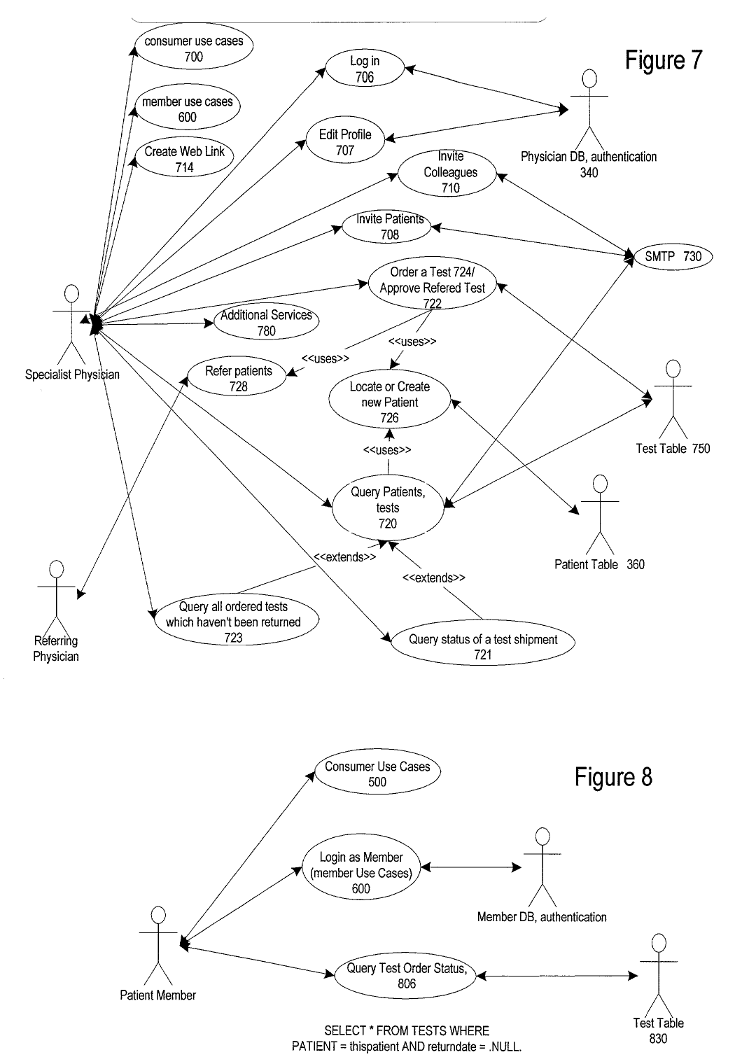

FIG. 2 shows an overview of the functional components of the system of the present invention, including a number of sub-systems 230, 240, 250, 255, 260, 275, 280, 290 (including both hardware, software and physical facilities) and classes of processes 207, 208, 211, 212, 214, 216, 217, 218, 219, 221, 222, 223, 224 used in the system. The system shown in FIG. 2 may be used to implement the general process of the present invention shown in FIG. 7.

As shown in FIG. 2, two types of interactive mechanisms 202,204 may be utilized by physicians and patients in accordance with the system of the present invention. Telephone and fax devices 202 and a computer terminal 204 such as a terminal connected to a variety of public and private networks, such as the Internet, allow various levels of users to interact in certain types of processes with the system of the present invention.

The embodiment shown in FIG. 2 is organized to service a number of different types of patients, physicians and consumers. Each type of user may request different services, and are given different privileges, based upon access and a registration process used in the present invention.

As shown in FIG. 2, the types of services available to a user will depend on the manner in which the user accesses the system. Certain services (indicated by dashed line 205) are only available through a telephone or facsimile interaction. For example, telephone and fax-only processes 207 allow members to register and request services from the system via a telephone call center 230 through a human operator, or through a fax machine. Other types of services are available to members through Internet access, as indicated by dashed line 215. Such services include browsing an Internet website 212 adapted to provide specific information services directed to sleep apnea and related subjects, Website-building services 214, and accessing specific topic chat groups 218.

A third set of services, indicated by box 210, are services which may be accessed via either telephone or fax 202 or Internet 204. These services include registering as a member of one of several privilege levels 211, ordering tests and other services 216, and reviewing data collected during tests 222.

Members are those users who have chosen to register with the system and are allowed to access additional services (including secure data interchange) which are not provided to non-members. Registration to become a member may occur through telephone, fax or Internet access. Non-members are allowed a lower level of access to information via the telephone, fax or Internet. If a member does not register via the web, the member may not be provided with a web-access account. Members may be both individuals (such as physicians) who order tests, and members who are to be tested (such as patients). Particular membership and user classes as pertaining to medical testing will be described in further detail below.

To handle telephone and fax processing services 205, the telephone and facsimile call center sub-system 230 may be staffed with human operators who receive calls from individuals and members interested in accessing system services, is provided. This call center sub-system may also comprise an automated data entry system.

Calls from individuals wishing to register as members are handled through call center 230 by “registering as a member” process 208. Calls from physician members who are authorized to order tests are handled by “Authorized requestor ordering service” process 219 through call center 230.

A database sub-system 275 is used in the system of the present invention to store member data, non-member data, test tracking data and other information. It may be maintained at the same physical site, or at a physically separate site, from the call center 230, or may be distributed between physically separate sites. Further details on the database system of the present invention will be described below. It will be recognized that the database system 275 handles all administration and management functions of the system of the present invention. Database system 275, in concert with call center 230 provides the authorization checking of decision block 103 (FIG. 1 B) for non-web access to the system.

A web-hosting server 250 provides the backbone for Internet web-based service classes 210,215. The web server 250 is configured for and interacts with both the Internet, to allow for browsing of specific types of Internet news groups 255 provided in a conventional manner through a commercial Internet system administrator, and interface with data entry requirements of the database system 275 of the present invention, including a registration process 208, ordering process 219, and data review process 221. As should be readily understood, appropriate security measures are implemented between the web server 250 and database system 275 to prevent unauthorized tampering of the database 275 to insure that only those two sites interact. This can be accomplished with encrypted point-to-point SSL connections, certificate-based authentication, virtual private networks or combinations of these and other well-known means. Web hosting server 250, together with database system 275 provides the authorization checking of decision block 103 (FIG. 1B).

Thus, authorized individual members, with the appropriate privilege level, may view collected data and test results either through the call center 230 (typically via return fax), or through web hosting server 250 and “Members reviewing tests” process 222. Access to collected data is through “Authorized viewing of collected data” process block 221, which insures that anyone requesting access to data is authorized to see that data.

The database sub-system 275 further interacts with a device management sub-system. In one embodiment, the device management sub-system 240 may comprise a separate database at the same or a physically separate location (such as a shipping provider) which tracks device specific data on each of the data collection devices utilized in accordance with the system of the present invention. As will be described in further detail below, each test device is uniquely identified and tracked through the various stages of the testing process from the point at which the device is ready for distribution to a user to the point when data is removed and the device refurbished for testing in a subsequent testing operation. In one embodiment, this occurs through the use of a unique, external serial number.

A logistics sub-system 260 shown in communication with database 275 provides shipping services, which ensure that the devices are shipped to the location where the test is to be performed. The logistics system includes shipping, receiving, transfer, warehousing and other such services 217, and may be performed by a common courier service, with warehousing services, such as Federal Express® or UPS®.

Also shown is a receiving and testing sub-system 280. The receiving and testing system 280 accepts returning test devices, extracts test data and enters it into the database system 275. The receiving and testing system may comprise a contractor which receives devices which are returned, refurbishes the devices, and ensures that the data is extracted from the devices and entered into the database system.

Finally, a billing and accounting sub-system 290 may represent a financial database and/or accounting applications which ensure that orders tracked and implemented by the system of the present invention generate a revenue stream for the manager of the system of the present invention.

Sleep Apnea Test Device Delivery and Data Recovery Process

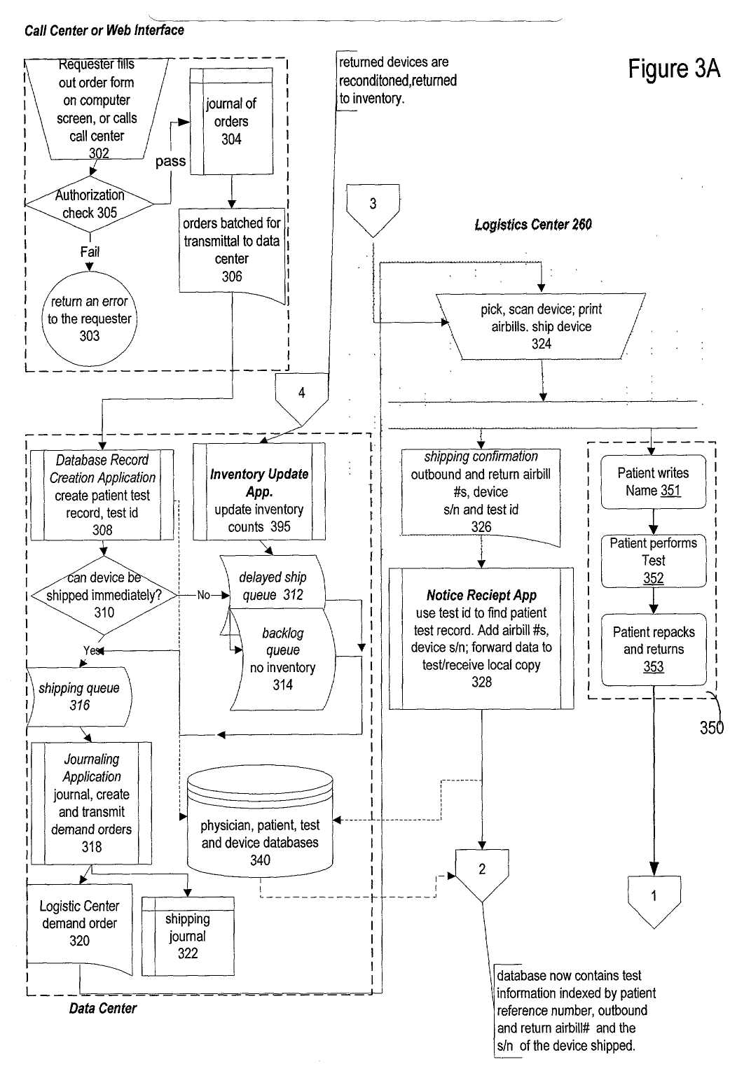

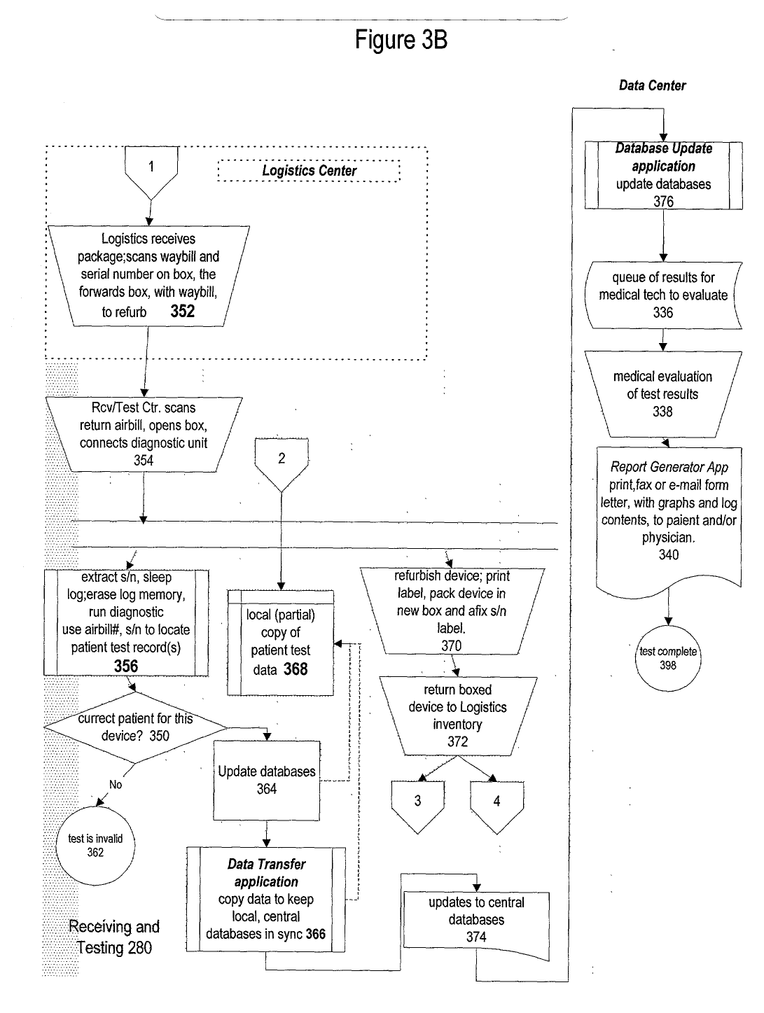

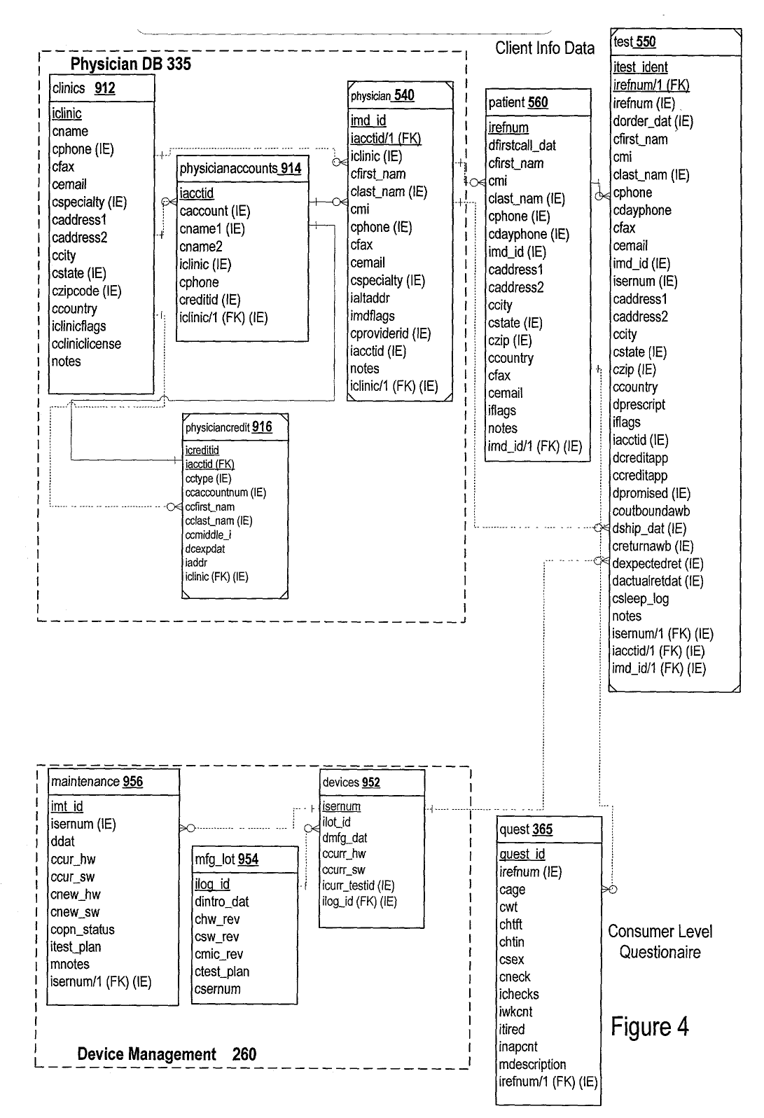

FIGS. 3A and 3B illustrate a specific embodiment of the method of the present invention wherein a sleep apnea test device is ordered and delivered to a patient. In the context of the following description, certain functions of the database and characteristics thereof will be described. An exemplary physical structure of the database will be described below with respect to FIG. 4.

In addition to database functions provided in the system, a number of data processing application programs will be discussed with reference to FIGS. 3A and 3B. Many of the programs relate to receiving messages and creating or updating database records assigning items to batch queues to be processed, taking items out of queues, and/or transmitting them to the shipping contractor or the refurbishing center contractor. In addition, applications for entering new devices into the database, editing the database to correct errors, running tests on devices, and updating the device records are described. There is also an application which examines test results and forwards reports to the correct medium (whether direct mail, fax, e-mail, or a secure web browser) to the physician or patient. While it should be generally understood that the applications are functionally distinct, in reality the applications may be integrated into one or more applications as may be convenient during system programming.

It should also be generally noted, referring to FIG. 2 from FIGS. 3A, 3B, the processes described in FIG. 2 are logical processes. The steps described in the flows of FIGS. 3A and 3B describe an implementation of, but do not correspond one-to-one with, the logical processes of FIG. 2. FIGS. 3A, 3B ignore the specifics of web browsers, web servers and how information is displayed and exchanged between them. These details are well understood by those of average skill in the art. Except where noted in FIGS. 3A, 3B with reference numbers from FIG. 2, the processes of FIG. 2 are implemented by steps in FIGS. 3A, 3B occurring at the data center. Embodiments of the present invention may have the data center steps all occurring at a single location, while other embodiments may have the data center steps occurring at physically separated locations.

The process begins at step 302 where a physician completes an order requesting a test. Orders are entered by telephone, or by a form submitted by fax, through the call center 230, or through the web hosting server 250 by filling out a form on a computer screen 304 and providing the requisite data for a patient. Following step 302, the ordering and call center sub-system verifies that the physician is authorized to order a test, and may also perform a funding or credit card verification check 305 (optional), before confirmation of the order. In one embodiment, following verification, an estimate of the initial ship date of the test device may be provided to the customer by querying an inventory of test devices and status maintained by system database 275. If authorization of credit checks in 305 fail, the order is rejected with an appropriate message to the physician.

Subsequent to the order authorization at step 305, the order is added to an order journal 304. In one embodiment, orders are batched for processing at a physically separate data center at step 306. A transaction batching process may be advantageous if, for example, the ordering and call center sub-system comprises a call center which is located physically separate from the data center. However, it should be recognized that real time processing of orders may be utilized, and the batching process indicated at step 306 may be eliminated, if real time communication between the system database and the order taking steps 302 and 305 is established. Alternatively, if step 302 occurs at a geographically separate call center, orders may be batched and transmitted periodically.

In one embodiment, orders may be entered into an audit journal 304 located with the call center, with the database 275, or elsewhere. Once the orders are accepted, the system can verify previous orders via acknowledgments for orders entered, and can return inventory information to the order entry application to allow the shipping estimates described above to be provided. Such acknowledgments may be used to “check off” orders in the journal, and erase order information from the database at the call center or web input center.

A database record creation application 308 receives orders and creates database entry for each such order. Each such entry includes a unique test ID, and also includes a unique identifier of the patient. Data entered includes the patient's name and shipping address. In certain embodiments, patient financial information may be captured to bill the patient for the service. In addition, a requested arrival date may be placed in the system. The system first determines at step 310 whether the test device can be shipped immediately. A check of an inventory count database 395 is made to determine whether inventory is available. If a patient or physician requests a specific arrival date, the order is placed in the delayed ship queue 312, to be shipped to arrive on the requested day. If the device cannot be shipped immediately due to a lack of inventory, the order is placed in the backlog ship queue 314. If there is no delayed arrival date, or if the arrival date requested arrives and inventory is available, the order is advanced to the shipping queue 316. Although not specifically indicated in FIG. 3A, a shipping confirmation may be returned to the ordering and call center sub-system 230 so that the journal entry for that particular order may be marked, and the order information erased from the order/call center application database.

In one embodiment, the logistics center 260 and receiving and testing sub-system 280 may be one or more physically separate facilities and separate from the call center sub-system facility 230, database system 275 and web hosting server 250. In such case, a journaling application which includes database 340 may be used to remove a batch of orders from the shipping queue 316, and send them to the logistics center 260 via a secure network interface suitable for exchange of electronic data interchange (EDI) data between the data center and the logistics center 260. Connections may be made to the secure network using a variety of media, including dial-up or Internet connections. The data center and logistics center 260 need not have the same connection media, nor do they need to create a direct connection between them, although such connection is contemplated within the scope of the system of the present invention. In another embodiment, a ship date or arrival date may be chosen by the ordering physician or patient, which is confirmed by the system after checking inventory and expected shipments and returns prior to the requested date. In another embodiment, the logistics center 260 is located at the data center with the database 340 and shipping queue 316, and the shipping queue is accessed by a simple query application. Devices are shipped from a small local inventory using a standard shipping application, such as Federal Express Ship It™, or a custom application.

Additional tasks performed by the journaling application 318 include creating a journal entry in a shipping journal 322 for transfer by the secure network and copying requests to and from the secure network for any traffic from the logistics center 260. In one embodiment, when the logistics center 260 receives an order, it will return a receipt acknowledgment of the order to the database 340. If such an acknowledgment is received, journal entries in the shipping journal 322 may be removed. Generation of the demand order 320 to the logistics center 260 is also handled by the journaling application 318.

Once the demand order 320 is transferred to the logistics center 260, at step 324 shipment of the device to the patient is arranged.

At the logistics center shipping desk, a shipping clerk may retrieve order information on a computer screen, select a data collection device from the inventory, scan the bar-coded serial number from the outside of the device's sealed shipping box, and print two waybills. One of the waybills is addressed to the patient for shipping the device out, the second is addressed back to the logistics center for the return of the device. The two waybill numbers are recorded with the test identifier in the database 340 to facilitate identification of the proper patient when the device is returned. Each waybill includes a test ID as the tracking number and the device serial number. The box also contains a piece of packing tape with which the patient may seal the box for returning to the test administrator following the study. A test device is selected from a pool of identical devices in a warehouse just prior to shipping in order to keep costs down. To facilitate this, the serial number is recorded electronically internal to the test device, and is also noted on a bar-coded label on the outside of the box. When a device is shipped to a particular patient, the waybills are printed with the patient's address and the test I.D., the device is selected and its serial number scanned and recorded with the patient and test I.D. This system of recordation, along with the waybill number and expected return time, ensures that the correct patient can be associated with the collected data after the test is completed. The steps involved in receiving a returned test device ensure that the correct serial number is recorded on the shipping box prior to placing the box in the warehouse.

Two processing sequences occur following actual shipping of the device at step 324. First, at step 350, the patient will receive the collection device and will perform the test 350 in accordance with the instructions provided with the data collection device, instructions from the ordering physician, or from the system administrator.

Secondly, at step 326 a shipping confirmation, which may include outbound and return airbill numbers, a device serial number, and a test identifier, will be returned to the data center and database 340. The serial number is recorded with the test record in 225, also the test I.D. may be recorded in 240 with the device record. Once the shipping confirmation 326 is received, a notice receipt and update application 328 is invoked at the data center to add new information, such as waybill number, ship date and serial number, to the record for this test in the patient, test and device database 340. In addition, the shipping request is removed from the transaction journal 322.

The notice of receipt and update application 328 also identifies the test record as having a test which is expected to be returned within a certain period of time. The period of time may be set at any number of days, but for a sleep apnea test and the present embodiment, an exemplary number of days is ten. In one embodiment, application 328 further sends information about the test (such as the device serial number, ship date, expected return waybill number and date, and test identifier) to the receiving and testing center 280 in FIG. 3B. In one embodiment, the receiving and testing center 280 includes a separate, partial copy 368 of the database 340 (FIG. 3B), which receives (via connector 2a) the copy of the aforementioned information. Another embodiment allows receiving and testing center 280 to access the central database 340 directly to access test information and insert collected data.

In one embodiment, physical test fixtures which couple to the returned data collection devices are used at the receiving and testing center to process returned data collection devices. The function of such units is described below, but at present it should be noted that a replicated database may be shared by all such test fixtures at the receiving and testing center, or replicated at each test fixture individually. If a standard commercial replicated database package is used, it will have its own mechanisms for replicating information which are scheduled to run periodically. If there is no replicated database, the same information may be extracted from the data management system and sent to the testing and receiving sub-system location (e.g. to the shared server or to each diagnostic unit), in a standard, searchable form, by application 328.

Returning to FIG. 3A, at sequence 350, once a box is shipped to the patient, the patient will unpack the box, write his or her name on a paper label affixed to the front of the device 350, and perform the study or test 352 which data collection device is designed to perform. This test may take several days and following the test, the patient will then put the device back in the box, seal it and use the return waybill to ship the box back 353 through the logistics system 260 to receiving and testing station 280. (It should be recognized that the patient's name is all that is required to track the test.)

Following connector 1 from FIG. 3A to FIG. 3B, at the logistics center at step 352, the package is received, the waybill number and the serial number on the box is recorded, and the box along with the waybill forwarded to the receiving and testing center 280.

Following the transfer of the package from the logistics center 260 to the receiving and testing center 280, at step 354 the return airbill is recorded, and the returned data collection device is removed from its shipping box and connected to the test fixture. The test fixture will check the basic operation of the device at step 356, and extract the internally-recorded serial number and data (sleep log) from the device. During the extraction step, the test fixture, which may comprise a personal computer with appropriate peripheral connections for a scanner and the test device, will read the serial number of the device and extract the sleep log from, for example, non-volatile memory and the test device. The serial number is stored in the file within the sleep log and is used to locate the device record in data management database 240. In one embodiment, a local copy 368 of data management database 240 is used, having been loaded with the correct device data by step 328. In another embodiment, step 356 accesses the master copy of the device management database 240 at the data center through a suitable data connection. In addition, the test fixture will erase the log memory and run a diagnostic to ensure that the device is functioning properly. The results of the diagnostic test may be permanently logged for documentation for entities such as the Food and Drug Administration (FDA).

Following the diagnostic, the test fixture, in step 356, will use the airbill number and serial number to locate the test record in 368 (for some embodiments) or directly in 340, 275 (for other embodiments). In decision box 350, checks are made to determine if the returned device contains data for the correct patient. In one embodiment, this check includes verifying that the test identifier in the device record, and the waybill locate the same test record. In an embodiment, the expected return date in the test record may also be checked to verify that the device is being returned when expected. An embodiment may further ask the technician operating the test fixture to verify that the name which the patient wrote on the paper label fixed to the data collection device (step 350) is the same name recorded in the test record. If the checks in decision box 350 pass, then there is a reasonable assurance that the sleep log for the correct patient has been found. If the checks at decision box 350 fail, a flag indicating that this test is invalid is placed in the test record in the database, and manual recovery is initiated.

If all the checks in decision box 350 pass, the patient record is updated with the sleep log at step 364 and the waybill number, and an indication is made that all checks were passed. At step 364, for one embodiment, this information will be used to update the local copy of the patient test data base 368 with the actual return date, the collected data, and specific information about the condition and operability of the returned device (whether the device was returned to inventory). The application 366, together with step 374, then synchronizes the local copy 368 with the master database at the data center. In another embodiment, these updates are made directly to the master database 340, 275, through a suitable data communications connection.

Because there is no guarantee that the box used to ship the device originally will be the box used to ship the device back, the waybill number accompanies the device box to the testing and receiving center facility 280. This provides an additional check on the testing procedure. In addition, the internally-recorded serial number is used, as a verification that the correct test has been received.

At step 362 if either the device and patient record is one which does not exist in the local copy of the database 368, or in the master database 340, 275, or an error was flagged earlier on either the device or test record, there may be ambiguity as to which patient and test this particular test belongs to. Procedures may then be deployed for resolving such errors.

Moreover, if the study period does not correlate with the waybill dates, then the device may have failed or the tracking process at the logistics carrier may have failed. If the outgoing waybill number does not correlate with this test, then the patient or health care provider may have more than one study occurring, i.e., they may have put the device in the wrong box. Each of these particular possibilities can be dealt with on an individualized basis.

In one embodiment, when the patient's test record is updated with a sleep log, the test record is placed into the queue at step 336 to be examined by a medical technician. At step 338, a medical technician can be provided with a display of the sleep report and perhaps an automated recommendation based on known factors which may result from characteristic compositions of known data points within the test parameters. For example, certain combinations of parameters may yield specific indicators pointing to a general conclusion for each combination. The technician is then allowed to select one of several form letters to be sent to the patient and the data management system may personalize it by inserting a name and address as needed. A report generation application 340 then allows the report to be generated and forwarded to the patient and/or physician through any number of different testing phases. In one embodiment, a form letter is chosen automatically when the log is analyzed, based on the results (e.g., the test was inconclusive, the test indicates that the patient has a problem, etc). Another embodiment may use the same queue 336 and evaluation step 338 for assuring the quality of the diagnostic testing service.

In one embodiment, queue 336 and step 338 are not performed, and step 340 generates the proper reports, storing them in the test record in database 340. These reports are available to authorized individuals through checks of process 221, and through the fax of call center 230, or web hosting server 250. At this point, the test has been completed at step 398.

Again returning to step 354, the testing and receiving center includes facilities for refurbishing test devices once they return to the testing and receiving sub-system. As shown at step 370, once a returned device has been diagnosed and the sleep log extracted, a refurbishing step 370 occurs. At this step, physical components of the device which are not reusable for sanitary or other reasons are replaced. The internally-recorded serial number is used to print a new label for the data collection device's shipping box, the device is packed in a new box and the new serial number label affixed. Subsequently, at step 370, the returned device is boxed and returned to the logistics center inventory at step 372. Following connector 4 from FIG. 3B to FIG. 3A, an inventory update application 395 at the data center updates inventory counts in the delayed ship queue and backlog 312 and 314, the function of which is previously described.

System Data Structure

FIG. 4 represents a depiction of the data structure in an exemplary database 340, and 275, 240, used in accordance with the present invention. Referring to FIG. 4, device management 240, comprising three tables, corresponds to device management 240 of FIG. 2. The remaining items in FIG. 4 correspond to database system 275 of FIG. 2. Collectively, the items in FIG. 4 correspond to database 340 of FIGS. 3A, 3B.

Database system 275 is, in one embodiment, housed at a physically separate location than the web system administrator or the call center local administration. Beginning at a point when a particular user decides to arrange for a test to be provided, a file must be maintained of the test where information may accumulate until it is reported to the user. This data management system is required for maintaining all such patient records since there is a large amount of records open at any one given time. As the test system progresses, information will be added to the record and the data management system quickly locates the patient record and a given number of related pieces of information are added to it. Within the constraints of the process flow, in one embodiment of the invention, the system uses a batch transaction model between locations of physically separate entities.

As shown in FIG. 4, the database will comprise a set of tables comprising what is collectively referred to herein as a physician database 335, at least one set of tables 550 collectively referred to as the patient database 560, an additional set of tables 750 which represent specific tests data collection services. In the current embodiment, this table contains one row or record for each medical diagnostic test ordered. In the current embodiment, the consumer questionnaire data table 365 contains data provided by members describing their sleep experience.

In one embodiment, the tables represented in the physician database include a clinics table 912, an accounts table 914, a credit table 916, and a physician data table 540. The physician data table 540 serves as the master data table for the physician database and includes identifications of the specific member data ID (IMD_ID), whether the physician is associated with a specific clinic (ICLINIC), items such as the first name, last name, middle initial, phone number, fax number, e-mail, specialty, specific doctor ID flags, provider ID's, account ID's, additional notes, and an alternative address data field. The clinic pointer will identify a specific clinic (ICLINIC) within the clinic's data table 912. Again, information such as name, phone number, fax and specialty, etc. will be provided in the data table 912. The physician's accounts data table 914 links directly to both the clinics table and the physicians table 925, and provides reference information to accounting personnel at the physician's office including the physician's ID account, contact names, phones, credit ID's, and the physician's clinic reference. Table 540 also contains authentication information, including username and password (encrypted) for authenticating the identity of a physician making a request. In the current embodiment, all physicians contained in table 540 enjoy the privileges of “physician” membership level. In another embodiment, different privilege levels may be encoded in table 540, for example referral privileges allowing a physician to refer patients and review the test results for referred patients.

The credit data table 916 contains credit card data information should the physician decide to pay for testing using a credit card, and can include credit information such as the account type, expiration, first and last name and billing information, as should be readily understood from an examination of the data table 916 in FIG. 4.

In the current embodiment, the patient info database comprises patient table 560, and includes data such as the first name, last name, a unique identification number (IREFNUM). In addition to contact information data, the table maintains the patient's current physician identification (IMD_ID).

Information in the patient table 560 may be referenced by the consumer-level questionnaire data table 365. The consumer-level questionnaire data table 365 includes a unique identifier (quest_id), a reference number (IREFNUM) referring to a patient in table 560.

Device Management 240 for an embodiment of the present invention comprises a device data table 952, a manufacturing lot data table 954, and a maintenance data table 956. The device data table includes information indexed on the unique serial number (ISERNUM) of the specific data collection device. For each device, a lot identifier, manufacturing date, the current hardware and software present in the device, and the unique identifier of a test (iCurr_TestID) referencing a record in test table 550.

The manufacturing lot identifier (iLot_id) references a separate table (mfg_lot, 954) indexed by manufacturing lot number. Table 954 records all information common to each manufacturing lot, including where individual components originated; over what time period devices in each lot were manufactured; the introduction date; the original hardware and software revisions; the current testing plan software revision (CTEST_PLAN) present in the device; and the serial number of the first device in this lot.

Finally, the maintenance data table 956 is linked through the serial number of the device (ISERNUM) to the device data table, and describes each and every thing which happens to that data collection device. Such information includes a date an operation was performed on the device (DDAT); the current hardware (CCUR_HW); the current software (CCUR_SW); whether hardware and software for the particular device was updated during the operation (CCNEWHW and CCNEWSW); the current operational status (COPEN_STATUS), testing plan which defines the last diagnostic operation performed on the device; and any notes provided by the test technician. Table 956 also records all items packed with the device and used in testing the patient, including: sensors, cables, plastic items, and manuals. Database 240 also comprises a device history file which is required by the United States Food and Drug Administration (FDA).

Other embodiments of the present inventions, for other kinds of data collection devices, will record different maintenance information, or may combine the function of maintenance table 956 with another table, such as test table 550.

Test data table 550 in the current embodiment describes each test performed using a data collection device. It is intended that one instance of this table, or a similar one, be provided for each service that an authorized requestor has ordered through the present invention. In the current embodiment, a record is created in table 550 by use case 724 (Order a Test) of FIG. 7, as described in application step 308 of FIG. 3A. The record includes substantially all of the information in the patient table 560 for the patient being tested, as well as a reference to the physician ordering the test. Step 328 adds the shipping information and serial number of the collection device shipped for the test. (This completes cross-referencing of tables 550 and 952.) Step 356 of FIG. 3B adds a copy of the collected data to the record of table 550 located by searching table 550 for the returned device's serial number. The check described for decision box 350 involves verifying that the unique record of table 952 returned when searching for the returned device's serial number, references a test identifier which in turn locates a test record in table 550, which itself contains the same device serial number as the returned device. Further, this test record must also contain the same waybill as was recorded in step 354, and the patient name in this record of table 550 must be the same as the name written on the device by the patient.

Data table 550 includes, for each test device, a test identifier (ITEST_IDENT); a unique identifier of the patient being tested (IREFNUM); the order date (DORDER_DATE). The record also contains the patient's first name, last name, middle initial, phone, day phone, fax, e-mail, a unique identifier for the patient's physician, serial number of the data collection device shipped to the patient, patient's address, and any applicable data flags. Further, the table 550 contains the dates when the test was ordered, the outbound waybill number was provided (COUTBOUNDAWB), the ship date (DSHIP_DAT), the return waybill number (CRETURNAWB), the date of expected return (DEXPECTEDRET), the actual return date (DACTUALRETDAT), and, as noted above, serial number and account ID information (ISERNUM/1; IACCTID/1). Some embodiments may also contain an account ID number and credit information.

In one embodiment, the test table 550 includes a copy of the test log from the retrieved device. In another embodiment, test table 550 also includes summarized test result data. In a third embodiment, the test log retrieved from the device, along with any summarization or analysis of the test log, is included in a separate table related to the test table by the test identifier TEST_IDENT.

The patient physician and test databases represent the relationship among patients and physicians and test records of each patient's test history. As additional services are expanded, additional tables may be added similar to the test table 550 shown in FIG. 4. Additional tables will be linked to the patient and physician tables as needed.

Logically, a record in test table 550 which tracks tests has information within it which is current at the time the test is ordered. The physician 335 and patient 560 databases contain information which is maintained up to date for active members by those members editing their profiles.

In sharing information between the diagnostic software used by a technician at the receiving and testing 280 and the data center, information is extracted from each individual database as needed and, for one embodiment as set forth above is copied to a separate read-only table and transferred to the remote location where devices are returned and received. In one embodiment, this information is accessible through an OBDC driver, such as the Microsoft OBDC driver. In another embodiment, another database access technique such as JDBC is used. Similarly, records in the device table 952 corresponding to devices in tests expected to be returned may be extracted from the device table 952 and transferred to the remote test location.

The diagnostic program at the remote location can use the information to verify the identity of the patient whose test has been returned. Maintenance records shown in FIG. 4 are created for the diagnostic operations performed on the device and appended to a maintenance table indexed by device serial number along with flags and text from all fields. Each night, or at some other regular interval, for one embodiment, the maintenance and test result tables are transferred to the device and the patient, physician and test databases, respectively, and appended to the corresponding tables as shown in FIG. 4. For another embodiment, those maintenance records are appended directly to the master device management database 260 using a suitable data communications means.

Virtual Test Lab

The system of the present invention allows for the provision of a “Virtual Test Lab.” This concept transfers the traditional real world sleep testing to virtual space.

In particular, the virtual test lab is a system and process by which a patient may be referred to a specialist for diagnosis and treatment. The treatment may be referred to the member by a member- or non-member physician. This referral is generally made by a primary care physician, but contemplates direct patient-to-specialist contact. The virtual test lab facilitates this diagnosis and treatment by allowing a physician who is a specialist in the particular area of concern to have the opportunity to browse referrals, decide whether to proceed with the test or not, and order tests for patients. The system provides total confidentiality of patient identity, medical data, and test results. The specialist can control which test referrals he or she may accept, the patients they wish to request tests for, and the number of tests they may interpret via the system.

With reference to FIG. 2, in one embodiment, the virtual test lab is provided by processes 223 and 224 within box 225. The primary care physician can enter referrals into the system directly from the web through “Refer patients for testing” process 223. The specialist can later enter the system and browse the referrals through “Approve referrals” process 223, choosing to accept or reject each of the referrals as a group or one at a time. If a specialist accepts the referral, an order will be entered through process 219, and a data collection device will be shipped to the patient. The collected data may then be analyzed by the specialist and optionally forwarded to the referring primary care physician.

In one embodiment, the member physician can provide limited privileges to the non-member referring physician, in web hosting server 250 and database system 275, to view results of tests for referred patients through the test review process 222, or through call center 230 and fax 202.

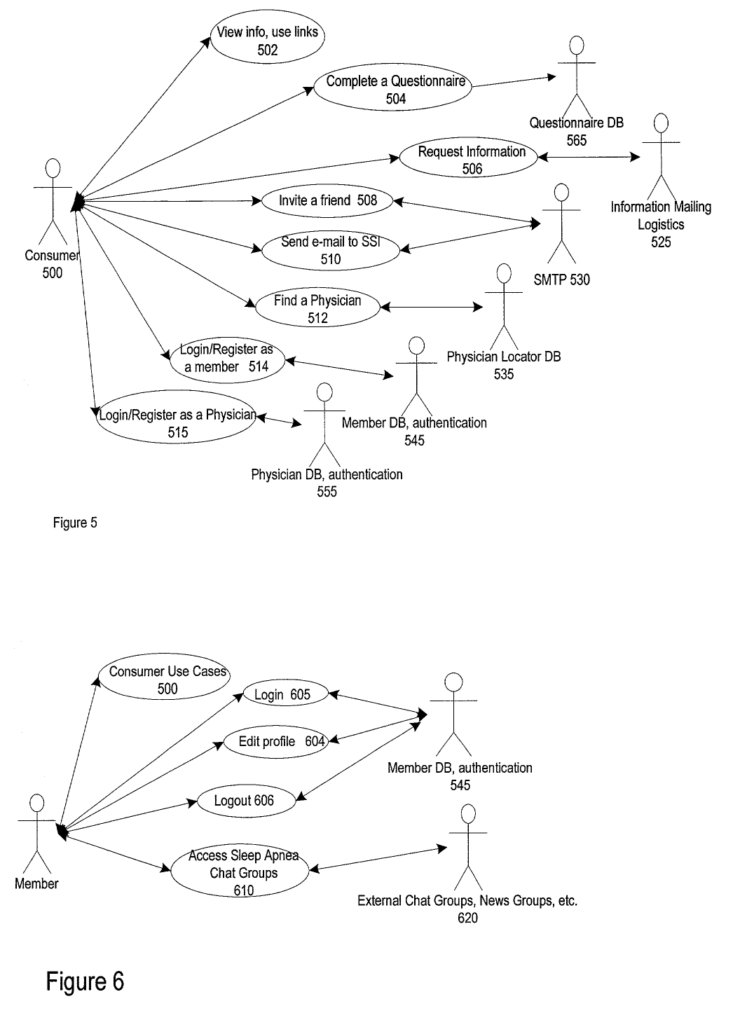

In accordance with the aforementioned embodiment, several levels of users are defined in the system of the present invention. FIGS. 5, 6, 7 and 8 are industry-standard Use Case diagrams specifying what ways each level of user may interact, in one embodiment, with the system of the present invention.

Non-Members

Non-member users include anyone in the general public who may wish to access specific information on the medical conditions the system administrator offers diagnostic services for, data about services, via the telephone or the World Wide Web. The ways in which a non-member may interact with the system are illustrated in FIG. 5. The non-member can view specific information concerning sleep apnea 502 (symptoms, causes, treatment alternatives, etc.), complete a questionnaire 504, or request information from the system administrator. A request for information may result in a request to mail information to the consumer via an information mailing action 525 or e-mail information via SMTP protocol 530. Likewise, non-members may further invite a friend 508 to join or visit the system's web server, or communicate directly with the system operator 510. This may be implemented by an e-mail form on the website or a mail to user and SMTP protocol 530. The non-member may use a find-a-physician service 512 which requires access 535 to a physician locator database 535 of physicians who have registered with the system and made themselves available for this service.

In addition, the non-member may choose to register or log in as a member. At member login, access to a member authentication database 345 is required. Finally, the non-member may register or log in as a physician member 515 requiring access to a physician authentication database 355.

As noted above, members are those individuals who have chosen to provide specific information about themselves and identify themselves in a system database in the system of the present invention. Registration allows members to access services which are available only to members. Privileges of various levels may be provided to members through a members-only website or via members-only functions available through the call center sub-system.

Members

Members-only access to particular functions is illustrated in FIG. 6. FIG. 6 shows uses of the system available to members who are not physicians. As shown therein, a member has full access to all of the consumer use cases 500 shown in FIG. 5. In addition, the member may login 605 and edit their profile 604, resulting in an interaction with the member database 345. In one embodiment, members may edit their profile to allow member physicians (or non-member physicians with privileges granted through the virtual test lab) to view their test results. The member may log out of the system 606, indicating to the system that no further privileged access may be made by this user until another login 605 is completed and may also access treatment-specific chat groups, such as sleep apnea chat groups, 610. Access to the sleep apnea newsgroups 610 may be maintained by the system administrator, or the system administrator may simply facilitate access to such chat rooms or newsgroups. If the system administrator does not maintain the newsgroups, the member is referred to an external chat group or news group host 620. Chat rooms may be hosted by the system administrator, or the system administrator may track who enters the chat room through the web hosting server portal as part of a service of providing additional servers and user-specific information for the members accessing the system.

Members generally fall into two categories: physicians and patients. Physicians can further be divided into specialist physicians and referring physicians. Physician members are the only members who can legally initiate a medical test. Registering as a physician requires completion of a number of verification processes. Verification is designed to ensure that a physician is licensed and requires the provision of specific information which allows the system administrator to verify the physician's license and standing to legally order medical tests. Only when verification is completed successfully is a physician admitted to this user class. After registering, but before verification is complete, a physician may request services, but those services will not be delivered until after verification is accomplished. All physicians must be members to access physicians-only services, either via the website or via telephone.

Privileged access to the current embodiment of the system of the present invention available to physician members is illustrated in the use cases of FIG. 5. As noted therein, a physician member has access to all consumer use cases which are represented in FIG. 5 (500), and all member use cases illustrated in FIG. 6 (600).

In addition, physicians may login (506), edit their own registration profile 507 and in doing so, interact with the physician database 355. Physician members may logout (508), indicating to the system that no further privileged access may be made until another login 506 is completed. Further, the physician can invite additional users to the system, including inviting a patient or consumer 508 or inviting a colleague 510. Doing so will invoke simple mail transfer protocol (SMTP) 530 as discussed above. An additional service provided to a physician is to create a link 514 to the physician's own website.

The physician has access to test report delivery and administrative functions, such as having test results forwarded at 516 via e-mail (including secure e-mail) again using SMTP 530. The physician is allowed to query certain test and patient information in the test records 550, by querying a patient's test records 518, querying the status of a test 520, or approving referring physicians' tests 522 if the patient has provided a medical release. Yet another option allows a physician to order a test 524 or locate or create a new patient 526 which includes interaction with a patient table database 560. A referring physician 500a may refer a patient for testing 528. The member physician 500 must approve the referring physician's test 522, before the system of the present invention will invoke its ordering functions. The virtual sleep lab described earlier is described by use cases 528 and 522.

Physicians who are members are also provided with an option of being displayed by a “find physician” service. Physicians must give explicit permission, by editing their profile 507 before their identity is released to anyone seeking a local physician.

In addition, member physicians may have the opportunity to access additional services 580 provided by the system administrator including: access to detailed information about sleep apnea tests and devices available through the service of the present invention, tutorials or classes for credit on sleep apnea 581, instructions in how to interpret test reports, and other information. Member physicians may further be provided with the option of building their own website 514 within the system administrator's website.

Still further, the member physician may be provided with the ability to host a virtual sleep lab within their own website, allowing users to access the information of the system administrator via the physician's website.

With respect to reviewing test results, a physician may be provided with various ways to select and view patient tests, such as selecting tests which have not yet been reviewed 523, with the system filtering tests by the physician's identity (that is, only tests ordered by that physician are selected for display), and the physician may select a specific test by patient personal information 520. The physician can provide some combination of name, e-mail address, approximate test date or other personal information for the patient in a query form or to an operator at the call center sub-system, and the system will attempt to locate the unique patient matching all the criteria listed. Alternatively, if the selection cannot locate a unique patient, all matching patients may be listed and the physician may choose the specific patient he wants or provide additional criteria to narrow the search. Once again, the physician's identity implicitly limits the search data so that the only tests returned are for patients who have explicitly released their medical records to the requesting physician by, for example, editing their member profile 604. In one embodiment of the present invention, these queries return data from the database table containing test information (550).

It should be particularly noted that unless a patient has specifically released their medical records to a requesting physician, those records are not visible to that physician. Even though the physician may be the most recent physician to order a test for the patient, the physician will only see items they ordered, unless the patient releases information to them. Releasing data to the current physician is done by checking the medical release box on the patient or member's profile 604. The authorization policy for viewing collected data, and checking for authorization, is performed by process block 221 of FIG. 2. As an example, consider a patient X who begins with a physician A. Physician A receives the sleep questionnaire. Then patient X goes to physician B who orders a sleep test. Then patient X goes to physician C who reviews the test and recommends treatment. If the patient has edited their profile and checked the box to release medical information to the current physician, physician C may see the entire history, physician B may only see the sleep test results, and physician A may only see the questionnaire. If patient X does not check the medical release box, then physician C may not see any of the patient's records. If C orders a test for X, then C may review the result of the test only until the patient checks the release box.

In addition, a physician will be offered the opportunity to be listed in the system administrator's “find a physician” directory. Those who provide such explicit permission, by editing their profile 507, will have the contact information displayed when a consumer asks for a physician in the physician's area. This service may be accessed through the Internet 204 or through the call center 230. Such a service will, for example, search on postal zip code and/or telephone area code.

Patient Members

Patients are individuals for whom a medical test has been ordered. Patient records in the system are “created” by a physician and associated with that physician in use case 526, usually as a side effect of ordering a test 524. For each patient, one or more tests, surveys, or data collection mechanisms may exist, and other information may be created. A user who registers as a member (FIG. 6), and who later has a test ordered for them becomes a patient through use case 526. His or her member profile acquires the privileges of a patient member in this way.

For example, a physician may order a sleep test with the system of the present invention, retrieve the results, then perform an outpatient corrective procedure in his office. Through the system the physician may order a follow-up test to assess the effectiveness of the procedure. In this case the physician will want to review the “before and after results” to assess the effectiveness of the procedure. The system administrator can, in such cases, provide additional information and procedural services, such as sleep apnea questionnaires, health assessment surveys, or other tests. Each instance of these services must be separate and associated with one patient and at least one physician. (Tests and procedures may be associated with more than one physician, such as the patient's primary care physician and the specialist who ordered the test, or with specialists of differing descriptions (such as a sleep specialist and a cardiologist)).

Disclosed patients are members who have allowed a medical release of their current information to their current physician. In requesting this service, the member must provide the physician's name, address and telephone number. In one aspect, the system administrator may forward the patient demographic and medical information to the physician with a cover letter. If the physician is a member, the patient record will be linked to the physician's by an identifier placed in the patient database record for the physician. Only disclosed patient records may be released to the current physician. If the patient has not also explicitly registered as a member, the patient cannot release their records and the requesting physician can only see the results of the test he or she ordered. To facilitate the registration process, registration may take the form of provision of a faxed medical release which provides access to designated physicians.

When a physician orders a test for a patient, and includes the patient's e-mail address, the patient will be sent a message confirming the test and the expected delivery date. The confirmation will include an invitation for the patient to become a member. If the patient is already a member as shown in FIG. 6, the patient has the ability to query test status 606, which interacts with the test table 550. In one embodiment, the patient may query the status of the test through web-access only, using a test confirmation number included in the confirmation e-mail. In a further embodiment, the patient may be allowed to review their test results and receive physician feedback via the site.

Virtual Test Lab—Physician Site

When a member physician creates a website or links a website to the system of the present invention, he or she may create a virtual test lab, as a page on their website. This allows the member physician to appear to his referring physicians as a sleep testing lab. Physicians may refer patients to the member physician through the virtual test lab page. The member physician can use the system of the present invention to administer the test, interpret the test results, and provide the test results back to the referring physician.

A virtual test lab page may be a variant of the form used by the member physicians to order tests in the embodiment shown in FIG. 2, process blocks 219. The virtual test lab form on the physician site, through process block 223 of FIG. 2, interacts with the system database 275 in a way that parallels the member physician's order entry process. The Universal Resource Locator (URL) to the virtual test lab page may be published on the physician's web page if he or she so desires. Alternatively, the member physician may choose to provide the URL only to physicians that he or she has agreed to take referrals from. As the member physician is financially responsible for all tests approved by him or through her virtual test lab, this makes it more difficult for the site to be compromised or for patients to order their own tests by entering their physician's name.

Referring Physician

A referring physician refers patients to member physicians (who are specialists) for testing. A referring physician is not a member and may not be tracked by all embodiments of the system of the present invention. In another embodiment, referring physicians' information may be tracked by the system of the present invention, and limited privileges provided to them. In another embodiment, “membership” to a physician's virtual test lab may be managed on behalf of the member physician, allowing the physician to provide his/her referral network with their own password access to his virtual test lab. When a member physician orders a test for a referring physician's patient, the referring physician generally need not be sent a confirming e-mail. However, notification mechanisms for referring physicians may be implemented.

The many features and advantages of the present invention will be apparent to one of average skill in the art. All such features and advantages are intended to be within the scope of the invention as described herein by the written description and figures, and as defined by the following claims.

In the present invention, the ordering call center sub-systems, local data system sub-system 240, logistics sub-system 260, and the receiving and testing sub-system 280, are contemplated as being physically separate from each other. However, all physical combinations and sub-combinations of the physical arrangement of the system are contemplated. For example, all sub-systems may be housed at a single facility. Alternatively, the logistics sub-system 260 may incorporate the receiving and testing sub-system 280; the database system, and ordering and call center sub-systems may be combined in a centralized data center and so on.

The logistics center shown in FIG. 2 may comprise, for example, a Federal Express® hub, and services may be contracted out to Federal Express® and/or UPS®. Certain interactions must occur between the logistics hub and the data system, including the issuance of a demand order to the logistics center, an acknowledgment of the demand order and a ship acknowledgment, an acknowledgment when a test device is returned from the user to the logistics center, and a testing routine wherein, if the device is not returned by the expected date, action is taken on the part of the logistics center and/or the data management system to retrieve the device.

If logistics center 260 comprises a Federal Express® hub, the data system and the communications infrastructure of the system of the present invention must be adapted to communicate with the logistics environment. Since an interface between the system of the present invention and the logistics center can be easily implemented on a batch-oriented electronic data interchange (EDI) transaction model, all communications may likewise be oriented around a batch model. However, if the logistics center 260 can accept real time transactional data in the EDI model or a separate model, a full-time connectivity and real time transaction data passage can be implemented in accordance with the system.

Electronic data interchange format through Fed Ex Net (Federal Express® value added network) is at least one particular format for interacting with the logistics system in accordance with the present invention. Test devices in accordance with the present invention may be kept in inventory at the logistics center hub sealed in boxes and ready to ship. The logistics center can receive demand orders electronically from the database system 275 and can reply with a confirmation or shipment and actual waybill numbers and device serial numbers. This is generally accomplished using standard EDI transactions.

In one embodiment, the receiving and testing sub-system 280 may be housed within the Federal Express® hub or logistics center hub itself, or may be a separate physical facility from the hub. This may be handled by an outside contractor when necessary. The work done at the receiving and testing sub-system 280 will be specific to the particular device. As noted above, equipment and software necessary for performing a cleaning and testing function on particular devices is adapted for each test device.

The many features and advantages of the system of the present invention will be apparent to one of average skill in the art. All such features and advantages are intended to be within the scope of the invention as defined by the written description and drawings presented herein, and in accordance with the following claims.

What is claimed is:

1. A system for delivering an in-home test device to a patient at the request of a physician, comprising:
a user interface coupled to a network;
a test ordering system coupled to the user interface;
an order verification system;
a database including patient, test and physician records;
a shipping and retrieval system, coupled to the ordering system, delivering the test apparatus to the patient; and
a result delivery system, delivering test results to the physician.

2. The system of claim 1 wherein the test ordering system comprises a call center.

3. The system of claim 1 wherein the user interface system comprises a World-Wide Web based interface.

4. The system of claim 1 wherein the shipping system comprises an interface to a commercial courier service.

5. The system of claim 1 wherein the verification system is housed at a physically different location from the user interface.

6. The system of claim 1 wherein the test ordering system includes a tracking system allowing authorized access to the test device status.

7. The system of claim 1 wherein the verification system includes an authorization routine interfacing with the database.

8. The system of claim 1 wherein the verification system includes a user security hierarchy

9. The system of claim 7 wherein the shipping and device retrieval system includes an interface to the database transferring shipping information to the database.

10. The system of claim 1 further including a device return verification application, communicating with the logistics system.

11. The system of claim 1 further including a data report generator.

12. A system for delivering a home test unit to a patient upon the request of an authorized physician, comprising:
a test ordering system, including a telephone access system, a facsimile access system and a WWW access system;
a data management system coupled to the ordering system;
a test distribution and retrieval system coupled to the data management system; and
a test data distribution system, coupled to the data management system;
wherein the data management system further includes:
an authorization system verifying the identity of the authorized physician;
an order tracking system; and
a device tracking system.

13. The system of claim 12 wherein the ordering system includes a call center subsystem.

14. The system of claim 12 wherein the data management system includes at least one database containing patient and physician records.

15. The system of claim 14 wherein the at least one database includes device tracking information.

16. The system of claim 14 further including at least a second database separate from the first database containing a subset of records of said at least one database, and physically housed at a common courier service.

17. The system of claim 12 wherein the data management system includes a security application defining and identifying different levels of users accessing the system.

18. The system of claim 17 wherein the user levels include members and non-members.

19. The system of claim 18 wherein the user levels include patient members, patient non-members, consumer members, physician members, and physician non-members.

20. The system of claim 14 wherein the test distribution and retrieval system includes use of a common courier.

21. The system of claim 20 wherein the system includes an interface application between the at least one database and the common courier service.

22. The system of claim 21 wherein each test unit includes a unique identification code which is exchanged between the courier service and the interface application.

23. The system of claim 12 wherein said test data distribution system includes a result retrieval system coupled to the test device when the test unit is with the patient.

24. The system of claim 12 wherein said test data distribution system includes a result retrieval system coupled to the test device when the test device is returned to the distribution and retrieval system.

25. A method for delivering a test unit to a patient and distributing results of the test, comprising:
(a) receiving an order from a physician for distribution of a test to a patient via a computer coupled to a network;
(b) verifying the authorization of the physician to place the order and the patient to receive the order;
(c) storing tracking information regarding a test unit shipped to the patient;
(d) shipping the test unit to the patient;
(e) retrieving test results from the unit; and
(f) distributing the test results.

26. The method of claim 25 wherein said step (a) includes receiving an order via a telephone.

27. The method of claim 25 wherein said step (a) includes receiving an order via a secure World Wide Web interface.

28. The method of claim 25 wherein said step (a) includes receiving an order via a facsimile.

29. The method of claim 25 wherein said step (c) comprises providing a database including a test unit identification number and patient information, and correlating the test unit information number and patient information during shipment and retrieval.

30. The method of claim 29 wherein said step (c) further includes storing, in said database, shipping information including a transmittal date to said patient.

31. The method of claim 25 further including a step, following said step (d), of retrieving the test unit from the patient.

32. The method of claim 25 further including a step, following said step (e), of retrieving the test unit from the patient.

33. The method of claim 25 wherein said step (f) comprises:
(f1) determining a user authorized to receive the test results and transmitting the test results to the authorized user.

34. The method of claim 25 wherein said step (f) comprises transmitting the test results via a secure World Wide Web connection.

35. The method of claim 25 wherein said step (f) comprises transmitting the test results by facsimile to an authorized recipient.

36. A system for delivering a medical test to a patient and distributing the results of the test to a physician, comprising:
a World-Wide Web based portal including a secure test ordering interface;
a database order processing system coupled to the portal;
a logistics system interface; and
a result distribution system.

37. The system of claim 36 wherein said order processing system includes a database including physician, patient, test and device records.

38. The system of claim 37 including a database record creation application adding said records to said database.

39. The system of claim 36 wherein said logistics system interface includes a shipping demand order output.

40. The system of claim 36 wherein said logistics system interface includes a receipt application input accepting shipping numbers, device ID number, and a test identifier number.

41. The system of claim 36 further including a receiving and testing system accepting returned test units.

42. The system of claim 41 wherein said receiving and testing system includes a refurbishing facility.

43. The system of claim 36 wherein said result distribution system includes a verification sub-system to determine authorized recipients for test results.

44. The system of claim 43 wherein the result distribution system includes a report generator.

45. A World-Wide Web-based portal interface to a system for delivering a medical test to a patient and distributing the results of the test to a physician, comprising:
a secure test ordering interface; and
a secure test result retrieval interface.

46. The portal of claim 45 wherein the secure test ordering interface is coupled to a system for delivering a medical test to a patient including a shipping system coupled to the secure test ordering interface; a verification system coupled to the secure test ordering interface; a retrieval system; and a verification system.

47. The portal of claim 46 wherein said secure test result retrieval interface is coupled to a result delivery system, which includes an authorization application and a patient and physician database.
